(19) United States
(12) Patent Application Publication
(10) Pub. No.: US 2018/0160959 A1
(43) Pub. Date: Jun. 14, 2018

(54) MODULAR ELECTRONIC LIE AND EMOTION DETECTION SYSTEMS, METHODS, AND DEVICES

(71) Applicants: Timothy James Wilde, South Lebanon, OH (US); Keyrsten Suzanne Wilde, South Lebanon, OH (US)

(72) Inventors: Timothy James Wilde, South Lebanon, OH (US); Keyrsten Suzanne Wilde, South Lebanon, OH (US)

(21) Appl. No.: 15/836,863

(22) Filed: Dec. 9, 2017

Related U.S. Application Data
(60) Provisional application No. 62/433,107, filed on Dec. 12, 2016.

(51) Int. Cl. A61B 5/16; G06K 9/62; G06F 15/18; G06K 9/00

(52) U.S. Cl. CPC A61B 5/164; G06K 9/00335; G06F 15/18; G06K 9/6267

(57) ABSTRACT

A modular, electronic lie and emotion detection system is disclosed. The modular, electronic lie and emotion detection system can include a computing unit programmed for multifactor dimensionality reduction, anomaly detection, and prediction of lies and emotions and may also be configured to communicate with the modular and/or remote unit(s) via an interface or port or connector to which the computing unit and the modular and/or remote unit(s) are coupled. The modular and/or remote unit(s) can supplement the functionality of the computing unit.

### BACKGROUND

### Field

The present invention described herein relates generally to adaptive lie and emotion detection systems, methods, and devices. In particular, some embodiments described herein relate to electronic communication systems and devices that can be used during human-computer interactions for automated lie and emotion detection.

### Background

There are numerous instances in which the ability to have accurate lie detection, coupled with an ability to determine emotional state, can be beneficial. Within the Medical domain; engaging patients to adhere and follow a clinical pathway is one of the toughest challenges in the industry . . . having an easily accessible determination of adherence would be of benefit. Within law enforcement, the ability to save a suspect from going to jail because they were incorrectly accused or determine the validity of witness testimony and truthfulness during depositions, could be of great value. Customs, Border Patrol, and Immigration could use such a system during interrogations to accurately determine a subjects involvement in terrorism, smuggling, or their desire to harm a country's citizens. In the private sector, criminal and civil incidents could benefit from such a system and the methods by which businesses interact with their employees could be positively impacted, with leadership able to understand the reactions of announcements, etc. upon their workforce, in addition to a more reliable practice of interviewing potential candidates. Another potential envisioned benefit of such technology is with real-time Focus Group assessments or in gauging an entire theatres emotional reaction to movie scenes, etc. Furthermore, an individual's personal life could be positively impacted, with reliable methods of understanding spouses, significant others, children, etc. truthfulness and emotional state.

With today's rapid technological innovations, current practices for lie detection have become outdated and are inherently invasive in nature, with little to no ability to be utilized in a mobile setting. These include the polygraph test (created in 1921) which by public opinion is widely considered to be unreliable, and the fMRI, which is expensive and impractical for day to day lie detection due to its large size and invasive nature. Methods of lie detection, which can have varying degrees of effectiveness when used exclusively, such as voice stress analysis, can aid in detecting lies in an individual, but are limited by their singular sensory inputs, and as such, are incomplete.

Lie and emotion detection can realize substantially increased system enumeration, efficacy, and reliability when combined into a homogeneous system utilizing a plurality of analysis and sensory systems, methods, and devices. However, challenges in the integration of multiple sensor inputs create hindrances in the ability to combine several inputs into one system. One such issue is that the expressions between the sensor outputs are not synchronized in many instances, another is that the very nature of human expression is inherently complex and difficult to decipher. Another issue is in differing mechanisms in interfaces and so on.

### SUMMARY

Accordingly, there is a need for improved lie and emotion detection and analysis electronic communication systems and devices. In some embodiments, interchangeable components, such as modular units, which can be utilized to supplement the capabilities of another component, such as a lie and emotion detection unit, can be implemented upon the electronic system or device. The lie and emotion detection unit can be, for example, a desktop computer, mobile device such as a laptop, smart phone or tablet, a helmet, or eyewear such as a goggle frame or an eyeglass frame, either with or without lenses. In some embodiments, the lie and emotion detection unit can include program(s) for integrating multiple sensory inputs and using them concurrently with the ability for the system to adapt with the introduction of further upgrades, or completely new technology.

In some embodiments, an electronic system is provided with interchangeable components, such as modular units, to supplement the capabilities of another component, such as a lie and emotion detection unit. The lie and emotion detection unit can include one or more components contained therein. The lie and emotion detection unit can in some embodiments include a processor within which can be memory, the process of which can be the storage and processing of data. The lie and emotion detection unit can also include one or more sensors, by which the system can obtain sensory data on the subject, the user and/or the environment. Receivers, transmitters, or transceivers can be utilized to communicate with other devices wirelessly, alternatively the ability to support wired connections through the use of ports and/or connectors allows the system to communicate via wired connections. This allows for the ability to connect modular units, as an example, including modular units such as but not limited to, an ambient or biometric scanner and a speaker. Other features can be performed by modular units, supplementing the capabilities of the lie and emotion detection unit.

In some embodiments, wired connections, such as a port and/or connector, can be included in the input/output system of the lie and emotion detection unit and electronic system. Included in this electronic system can be a modular unit. An input/output system, containing wired connections such as a port and/or connector can be utilized in this modular unit as well as a receiver, transmitter, and transceiver configured to wirelessly communicate with at least one remote unit, as well as the following components: a processor, a memory, and a sensor. Wired connections between the input/output system of the lie and emotion detection unit and the input/output system of the modular unit can occur when the two units are in a coupled configuration via a wired connection, allowing the transfer of data between the lie and emotion detection unit and the modular unit, and vice versa.

In some embodiments, a second wired connection can be included from the modular unit to another modular unit. By configuring the modular unit, between the second modular unit and the lie and emotion detection unit there can be communication allowing the transference of data between the lie and emotion detection unit and the second modular unit. Additionally, in some embodiments of the system wherein the lie and emotion detection unit includes a power source, the lie and emotion unit detection unit can power the second modular unit by utilizing a connected port and connector of the lie and emotion detection unit and the modular unit.

In some embodiments, the modular unit including an input/output system can include a receiver and a transmitter, the utilization of which allows the wireless communication between the modular unit and at least one remote unit, when configured to do so. The at least one remote unit can include a sensor. In some embodiments, the at least one remote unit can include a smart phone.

In some embodiments, two or more wireless protocols can be included in the receiver of the modular unit, such as but not limited to, ANT, ANT+, Bluetooth, Bluetooth Low Energy also known as Bluetooth Smart, MMS, Wi-Fi, CDMA, Zigbee, and GSM, and/or any other type of protocols which are similar to these and yet to be developed. The lie and emotion detection unit receiver can include one or more of the aforementioned protocols. Communication between the lie and emotion detection unit and/or the modular unit can exist in such a way that a first wireless protocol can be utilized between one remote unit, and a second wireless protocol be utilized between another remote unit. In some embodiments, an electronic system can include a lie and emotion detection unit, a modular unit, and an input/output system, with the lie and emotion detection system including at least one port or connector. Complimentary ports and connectors can exist on the lie and emotion detection unit and the modular unit, so that when connected in a complimentary fashion a connection will occur, and a wired electrical connection will occur. This connection can provide communication such as the transfer of data between the lie and emotion detection unit and the modular unit, and vice versa.

In some embodiments, the lie and emotion detection unit can include at least one of the following components: a processor, a memory, a sensor, a receiver configured to wirelessly communicate with a remote unit, and a transmitter configured to wirelessly communicate with a remote unit. In some embodiments, the modular unit can include at least one of the following components: a processor, a memory, a sensor, a receiver configured to wirelessly communicate with at least one remote unit, and a transmitter configured to wirelessly communicate with at least one remote unit.

In some embodiments, a receiver and a transmitter can be included in the modular unit and a processor can be included in the lie and emotion detection unit. One or more source devices, such as a modular unit or a remote unit, can generate a signal indicative of any one or more of the following; biometric information, sensor information, and/or other information pertaining to such signaling. Such devices as visual display, audio output, haptic feedback, or a combination of these transmission mechanisms can be utilized to transmit output to the user and/or subject, depending on the nature of the display and the preference of the user and/or subject in terms of display format.

In some embodiments, single function devices can be utilized which can be discrete and unique in nature. In other embodiments, a single device can determine and/or sense one, two, or three or more parameters. A plurality of source devices can be removably interfaced with the electronic system or device, while others may be wirelessly paired with the electronic system or device within the range of a network. In some embodiments, these may be worn by the user and/or subject or removably coupled to equipment (e.g., portable cart, vehicle, etc.)

In some embodiments, visual components, such as an image displayed to the user and/or subject, can be provided by the electronic system, either by the preexisting lie and emotion detection unit, or a modular unit and/or remote unit. In some embodiments, audio components, such as an audible signal perceptible by the user and/or subject can be provided by the lie and emotion detection unit, modular unit, and/or remote unit. In some embodiments, haptic components, such as a plurality of tactile feedback elements, can be provided by the lie and emotion detection unit, modular unit, and/or remote unit. The generated tactile signal can be perceptible by the user and/or subject from the lie and emotion detection unit, modular unit, and/or remote unit.

In some embodiments, the first questions asked the subject will be used to establish a baseline. The questions may follow a script, with the subject's answers informing the next path of questions that will hold the most relevance. The interview technique can in some embodiments follow a kinesic approach, which can allow the user to evoke reactions from the subject in a meaningful and beneficial manner. These questions will help identify certain individual actions the subject demonstrates, in particular those which relates falsehoods told by the subjects. The questions can also aid in establishing the thresholds for different inputs. For example, the user can ask about the temperature in the room which may adversely affect the use of thermal sensor systems.

In some embodiments, the subject will be interviewed multiple times. Previously recorded sessions can better inform upon the subjects individual reactions to situations and questions. The benefits of multiple sessions can be compounded, establishing several data points and allowing a narrower margin of error.

In some embodiments, multifactor dimensionality reduction will be utilized for input and feature selection from a plurality of sensors and commercially available systems inputs potentially in conjunction with data normalization methods and canonical-correlation analysis, etc. These processes can be utilized to produce a model equation for a plurality of explanatory measures and a plurality of performance variables, allowing the lie and emotion detection system to overcome the design challenges associated with the integration of different input modalities.

In some embodiments, the process of predictive modeling can begin by establishing the ideal weight vector for a session. The current weight vector will be established based on the different weights of each input and their relevance to the aggregated system. Several different algorithms can be chosen based on the machine learning phase and implemented herein. For example, naïve-bayes algorithms may be utilized to identify patterns of the subject behavior over time. The system shall detect and eliminate anomalies, which can be detected based on the incoming data and adaptably modified to utilize the best suited algorithm(s) for each use case needed.

In some embodiments, the lie and emotion detection unit can utilize the programs contained therein which allow the access to several overarching algorithms used in plurality to allow the system the ability to predict the presence of truth or deceit in a subjects response to questioning, as well as their emotional response to stimuli.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples of embodiments in accordance with the disclosure, and are not to be considered limiting of its scope, the disclosure will be described with additional specificity and detail through the use of the accompanying drawings.

FIG. 1 illustrates a schematic of an embodiment of an electronic system in communication with a modular unit and a remote unit.

FIG. 2 is a flowchart outlining the process of multifactor dimensionality reduction of sensor inputs and anomaly detection and elimination, and prediction of lies and emotions according to one embodiment.

FIG. 3 illustrates the visual display of devices of the lie detection system according to one embodiment.

FIG. 3A illustrates the visual display of devices of the emotional analysis system according to one embodiment.

FIG. 4 illustrates the process by which several embodiments of the lie and emotion detection system can interact on a network, through which a server can be employed for lie and emotion detection and analysis.

### DETAILED DESCRIPTION

The modular system as specified in the following illustrations demonstrates the specifications of an electronic device or system, such as automated lie and emotion detection systems. The following embodiments detail the usage of specific types of lie and emotion detection technology, such as thermography. However, it is to be understood that the specifications below are not meant to be limiting, and as such a unit described as integrated within the system may be actualized as a modular unit and/or remote unit, and vice versa. This equipment can be any sensor connected to a computing device. Additionally, these embodiments can be combined or fused with other embodiments when advantageous. The inclusion of any system, procedure, and or protocol within this document does not necessitate the inclusion of these systems, procedures, and/or protocols in the final system and can be altered with any other system, procedure, and/or protocol within this document. The inclusion of any embodiment should not be considered limiting, as such the system can omit any embodiment and still function as designed.

### General System

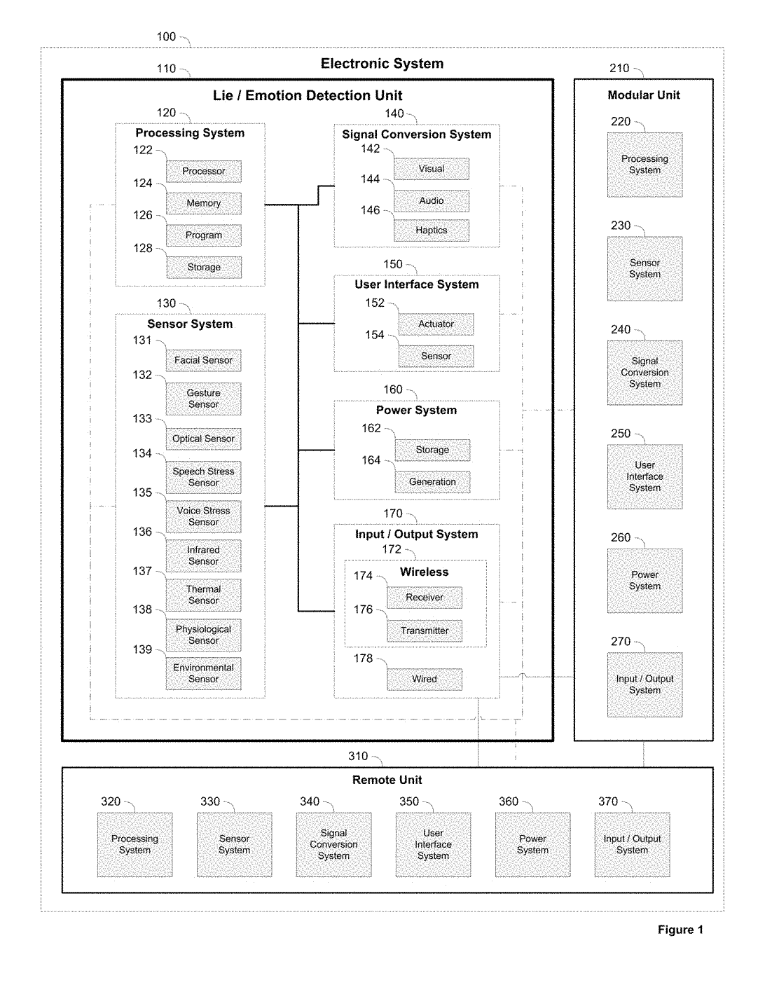

First in reference to the embodiment of the modular device or system 100, such as an automated lie and emotion detection system. In some embodiments, the automated lie and emotion detection system can include a computing system designed to receive input(s). As illustrated in FIG. 1, data captured from a sensor can be collected, processed and/or relayed to the system 100. The system can be designed as follows and include the following systems: a processing system 120, a sensor system 130 (consisting of but not limited to the following sensors: facial, gesture, optical, speech stress, voice stress, infrared, thermal, physiological, environmental, etc.) a signal conversion system 140, a user interface system 150, a power system 160, and an input/output (I/O) system 170. Modular unit(s) can be used in connection with the system 100, and be either wirelessly interfaced with the system 100 or connected to the system using wired couplings. In some embodiments, the primary function of the remote unit can be that of a camera, with the potential of additional capabilities. In some embodiments, a mobile device can be used to access the user-machine interface. In these instances, the user and/or subject may benefit from a more readily accessible and compact system.

As depicted in the illustration of one embodiment, each of the modular units can include one or more systems which can show similarities to the ones previously described. As such, the modular units 210 one or more systems such as a processing system 220, a sensor system 230, a signal conversion system 240, a user interface system 250, a power system 260, and an input/output (I/O) system 270 can show similarities with the lie and emotion detection units processing system 120, sensor system 130 (consisting of but not limited to the following sensors: facial, gesture, optical, speech stress, voice stress, infrared, thermal, physiological, environmental, etc.) signal conversion system 140, user interface system 150, power system 160, and input/output (I/O) system 170. In some embodiments, the system 100 modular units 210 and remote units 310 can be commercially available systems, methods and devices integrated into the primary system 100. For example, thermal imaging sensor(s) and system(s).

As the system is outlined, communication, wired and/or wireless, can be securely established between one or more of these systems. In some embodiments, communication between systems can be two-way communication and thus communication can be received and transmitted from each system to the other. For example, physiological data, such as that obtained by a FitBit, can be transmitted by the sensor to the system 100, which can process said data to aid in determining the presence of deceit. It can then transmit a signal back to the physiological sensor 138 which will provide the user and/or subject with haptic feedback when deceit has been detected. In some embodiments, the communications will be one-way communication, such that one system will transmit data and will be received by another system, but the system will not transmit data in return. For example, an environmental sensor 139 such as a thermometer can transmit data on the temperature of the subject's surroundings, while no data from the system 100 to this sensor is necessary. Any system discussed herein has the potential to engage in one-way or two-way communication with any other system discussed herein. Additionally, it should be understood that within the system as a whole, connections between systems can be implemented in such a way that any and all systems can be in contact at the same time, via direct and/or indirect connections. For example, mutual connections with the processing system 120 can allow indirect communication between the sensor system 130 and the user interface system 150.

In some embodiments, communications between the lie and emotion detection unit and one or more modular units can be established by either wired and/or wireless connections, such as via input/output systems 170, 270. Data may be transferred to the lie and emotion detection unit and one or more modular units and vice versa. In some embodiments, the modular units 210 may be connected via one-way communications such that data is transferred to the lie and emotion detection unit but not received, or vice versa. For example, a modular unit such as a thermal imaging camera can connect to the system 100 and provide feedback on its functionality as well as its readings, however no data need be transmitted to the camera in return. These connections between one or more modular units and the lie and emotion detection unit 110 can be established in excess of one, with some connections designated as one-way and others two-way communications. This can create situations wherein one modular unit may have two-way communications with the lie and emotion detection unit 110, whereas another modular unit may have one-way communications to the same lie and emotion detection system. Additionally, it should be understood that within the system as a whole, connections between modular units can be implemented in such a way that any and all systems can be in contact at the same time, via direct and/or indirect connections. For example, mutual connections with the input/output system 170 can allow indirect communication between a first modular unit 210 and a second modular unit 210, or direct communication can be established between the two units by utilizing the input/output system 270.

The depiction of an embodiment of this system as depicted in FIG. 1 demonstrates the connection between systems through the use of solid connecting lines. The utilization of the power system, such as by the lie and emotion detection unit 110 or modular unit 210, is demonstrated by the use of dash-dot-dot-dash lines. The utilization of power can be drawn solely from the power system 160 or supplemented by power system 260, 360. Alternatively the system can be supplied solely by the power system 260, 360. This depiction is also not meant to be limiting in relation to the use of the processing system in regards to communication, as the system may communicate directly, bypassing the use of the processing system 120 in some embodiments.

In some embodiments, the modular device may exist as an extension of the system, i.e. unable to be utilized as a separate standalone device. In these embodiments, the modular unit can exist without its own power system 260, using the lie and emotion detection units 110 power system 160 or another device.

In some embodiments, communication can be established between the lie and emotion detection unit 110 and/or the modular unit 210 with one or more remote units 310, utilizing either wired and/or wireless connections. As one embodiment is depicted in FIG. 1, the remote unit can include one or more such systems as the following: processing system 320, sensor system 330, signal conversion system 340, user interface system 350, power system 360, and input/output (I/O) system 370. As detailed in the following, the remote units 310 one or more systems such as a processing system 320, a sensor system 330, a signal conversion system 340, a user interface system 350, a power system 360, and an input/output (I/O) system 370 can show similarities with the lie and emotion detection unit(s) and/or modular unit(s) processing system 120, 220, sensor system 130, 230 (consisting of but not limited to the following sensors: facial, gesture, optical, speech stress, voice stress, infrared, thermal, physiological, environmental, etc.) signal conversion system 140, 240, user interface system 150, 250, power system 160, 260, and input/output (I/O) system 170, 270.

In some embodiments, the system 100 can be integral in the operation of the remote unit 310, alternatively the remote unit 310 can include systems which allow it to operate independently as a standalone device. The following list is an example of potential electronic devices which can be used as remote units, with the understanding that examples not listed herein can be utilized in a similar manner: PDA's, tablets, game consoles, microphones, cameras, cell phones, sensors, smart phones, laptops, smart watches, desktops, heads-up-displays, retinal projection devices, etc.

In some embodiments, data can be presented and/or communicated to the user and/or subject of the system 100 whereby the data is relayed from the one or more remote units 310 to the lie and emotion detection unit 110 and/or the one or more modular units 210. For example, the remote unit 310, such as a camera, can be utilized to capture and/or record additional data and be used in conjunction and/or additionally with the data collected by the sensors connected to the lie and emotion detection unit 110 and/or the modular unit 210 to provide a more precise and/or exact reading. In some embodiments, the lie and emotion detection unit 110 and one or more remote units 310 can be utilized without the presence of a modular unit 210, such as instances where the user and subject are able to access the same lie and emotion detection unit 110. In some embodiments, the lie and emotion detections unit 110 and/or one or more modular units 210 can interface with a remote unit 310, such as a smart phone, to allow for video conferencing with lie and emotion detection capabilities. In some embodiments, the lie and emotion detection unit 110 and/or the one or more modular units 210 are designed to utilize one remote unit 310, or can receive data from several connected remote units 310.

In some embodiments, the utilization of modular units 210 within the system 100 can allow a wide range of sensor inputs, which can increase the functionality of the system 100. This also allows the system 100 to implement new lie and emotion detection technologies as they are discovered and/or created, thus it is conceivable that the system 100 can exceed the current lifespan and viability of technology to allow for a more adaptive system 100.

### Processing System

Expressed in FIG. 1 is an illustrated embodiment of the system 100 such as a lie and emotion detection unit 110, which can be embodied by a unit including a processing system 120, the use of which can include storage of data and the processing of information from systems within the system 100, such as the lie and emotion detection units 110, the modular unit 210, and the remote unit 310 such as the aforementioned data. As expressed in the embodiment as illustrated, a processor 122, memory 124, program 126 and storage 128 are potential components to be included in the processing system 120. A microprocessor or a central processing unit, also referred to as a CPU, can be the method of which the processor 122 is realized. In some embodiments, the processor 122 can be capable of processing data through the utilization of one or more algorithms, either from the program 126 or servers of the system. The processed data can be further utilized and/or enhanced, or alternatively can be stored in memory 124 and/or storage 128 for future use. For example, this data, such as previous sessions, can be stored in the memory of the system and retrieved to implement in a current session, or to be further assessed by the user. In some embodiments, the realization of the program can be in the form of software which is stored in the memory 124 and/or storage 128 via firmware. The program 126 can receive updates and/or be modified for optimization in some embodiments by receiving new or updated programs, either by attaching the system via wired connections and/or wirelessly to a different computing system, attaching a new system, and/or by transitioning to a new system to replace an older and/or less viable system. The data to be processed can be received from one or more of the systems in the system 100, after which the data can be transmitted to one or more systems, or stored in the memory 124 and/or storage 128. The utilization of different programs 126 can enhance and/or alter the function of the processor 122 and/or any component of the lie and emotion detection unit 110, modular unit 210, and/or remote unit 310.

In some embodiments, software can be utilized with mobile devices as their source whereby the program 126 can be optimized, the list includes but is not limited to PDAs, smart phones, tablets and cell phones running iOS, Windows operating system, and/or Android, etc. A potential example of the aforementioned process is that the lie and emotion detection unit 110 can be designed in such a way as to include iOS, Windows operating system, and/or Android, enabling compatibility with like software. In some embodiments, software utilized in devices such as but not limited to desktops and/or laptops can be implemented in the program.

While in the previously alluded to FIG. 1 the program 126 is shown within the processing system 120, this is only one potential embodiment of this system. The program 126 can in some embodiments be firmware located in any other component of the lie and emotion detection unit 110, and can in fact be utilized several different ways in the same system. For example, the program 126 might be utilized to operate the components of the lie and emotion detection unit such as various components of the sensor system 130 (consisting of but not limited to the following sensors: facial, gesture, optical, speech stress, voice stress, infrared, thermal, physiological, environmental, etc.) the signal conversion system 140, the user interface system 150, the power system 160, and the input/output (I/O) system 170, as well as comparable systems on the modular unit 210 and/or the remote unit 310. This could include, for example, the operation and/or control of the wireless system 172 of the I/O system 170 such as the components of a transmitter, receiver, and/or transceiver. Typically, such connection are to be utilized within a network such as a LAN—Local Area Network, WAN—Wide Area Network, WLAN—Wireless Local Area Network, MAN—Metropolitan Area Network, SAN—Storage Area Network or System Area Network or Server Area Network or Small Area Network, CAN—Campus Area Network or Controller Area Network or Cluster Area Network or PAN—Personal Area Network, using wireless protocols such as, but not limited to, ANT, ANT+, Bluetooth, Bluetooth Low Energy also known as Bluetooth Smart, MMS, Wi-Fi, CDMA, Zigbee, and GSM, or alternatively connected by the network to the servers. Another usage of the program can be the monitoring of status, for example of the data being streamed from one or more sensors.

### Sensor System

Using FIG. 1 as an illustrated embodiment for a potential realization of the system 100, a sensor system 130 can be included, the primary utilization of which would allow the capture of sensory data from the subject (e.g., facial, gesture, optical, speech stress, voice stress, infrared, thermal, physiological) and/or the environment of the subject (e.g. environmental or ambient). The realization of this system can allow multiple sensors of but not limited to such natures as, one or more facial sensors 131, one or more gesture sensors 132, one or more optical sensors 133, one or more speech stress sensors 134, one or more voice stress sensors 135, one or more infrared sensors 136, one or more thermal sensors 137, one or more biometric and/or physiological sensors 138, and one or more ambient or environmental sensors 139, etc. The data collected and/or captured from the sensor system can allow the system 100 to gain insight into the subjects emotional wellbeing, for example it can allow the user insight into the subjects reaction to news as told by the user or a third party, which can inform the users next course of action. The sensor system 130 can also give insight into the subjects use of deception while conversing with the user, a third party, or while exclusively monologuing, for example while reading a prepared statement. Environmental sensors 139 can allow insight into the setting of the subject, for example the ambient noise could suggest that the subject is in a populated setting. This further information affords the ability of the system 100 to utilize the sensor data to the highest contribution level possible.

In some embodiments, the one or more facial sensors 131 can be designed to track, measure and/or detect motion, action, activity and/or movement sensors which can be encompassed by this classification include those which can perform functions such as, but not limited to, movement of the muscles in the face. Another example of this classification of sensor can be, but not limited to, identifying movements in the face which provide insight into a subjects unique psyche, such as the identification of an individual expression, or a “tell”. The data from this sensor may be collected from a facial sensor 131 upon the lie and emotion detection system, as well as solely or in conjunction with sensor systems 230, 330 connected via modular unit 210 or remote unit 310.

In some embodiments, the one or more gesture sensors 132 can be designed to track, measure and/or detect motion, action, activity and/or movement. Sensors which can be encompassed by this classification include those which can perform functions such as, but not limited to, changes in the subjects posture. Another example of this classification of sensor can be, but not limited to, a sensor for tracking a subjects hands as they speak. The data from this sensor may be collected from a gesture sensor 132 upon the lie and emotion detection system, as well as solely or in conjunction with sensor systems 230, 330 connected via modular unit 210 or remote unit 310.

In some embodiments, the one or more optical sensors 133 can be designed to track, measure and/or detect motion, action, activity and/or movement. Sensors which can be encompassed by this classification include those which can perform functions such as, but not limited to, tracking the movement and dilation of a subjects pupils. Another example of this classification of sensor can be, but not limited to, tracking gaze fixation of the subject. The data from this sensor may be collected from an optical sensor 133 upon the lie and emotion detection system, as well as solely or in conjunction with sensor systems 230, 330 connected via modular unit 210 or remote unit 310.

In some embodiments, the one or more speech stress sensors 134 can be designed to track, measure and/or detect audio patterns. Sensors which can be encompassed by this classification include those which can perform functions such as, but not limited to, irregularities in the cadence of the subjects diction. Another example of this classification of sensor can be, but not limited to, a sensor for tracking the times a subject retracted or corrected their verbiage. The data from this sensor may be collected from a speech stress sensor 134 upon the lie and emotion detection system, as well as solely or in conjunction with sensor systems 230, 330 connected via modular unit 210 or remote unit 310.

In some embodiments, the one or more voice stress sensors 135 can be designed to track, measure and/or detect audio patterns. Sensors which can be encompassed by this classification include those which can perform functions such as, but not limited to, microtremors in a subjects voice. The data from this sensor may be collected from a voice stress sensor 135 upon the lie and emotion detection system, as well as solely or in conjunction with sensor systems 230, 330 connected via modular unit 210 or remote unit 310.

In some embodiments, the one or more infrared sensors 136 can be designed to track, measure and/or detect radiant energy. Sensors which can be encompassed by this classification include those which can perform functions such as, but not limited to, increases in oxygen levels within blood on the face due to increased brain activity. The data from this sensor may be collected from an infrared sensor 136 upon the lie and emotion detection system, as well as solely or in conjunction with sensor systems 230, 330 connected via modular unit 210 or remote unit 310.

In some embodiments, the one or more thermal imaging sensors 137 can be designed to track, measure and/or detect infrared radiation. Sensors which can be encompassed by this classification include those which can perform functions such as, but not limited to, blood flow analysis, for example, to track the flow of blood around the subjects eyes. The data from this sensor may be collected from a thermal sensor 137 upon the lie and emotion detection system, as well as solely or in conjunction with sensor systems 230, 330 connected via modular unit 210 or remote unit 310.

In some embodiments, the one or more physiological sensors 138 can be designed to track, measure and/or detect physical and/or bodily parameters and/or responses of the subject. Sensors which can be encompassed by this classification include those which can perform functions such as, but not limited to, a sensor which can track changed in the subjects perspiration, galvanic skin activity response, skin conductance, sympathetic skin response, electrodermal activity responses, cardiovascular changes such as blood pressure sensor and heart rate sensor, a sensor that can track the subjects overall body temperature, etc. The data from this sensor may be collected from physiological sensors 138 upon the lie and emotion detection system, as well as solely or in conjunction with sensor systems 230, 330 connected via modular unit 210 or remote unit 310.

In some embodiments, the one or more environmental and/or ambient sensors 139 can be designed to measure and/or detect the surroundings that the subject is located in while the system 100 is in use. Sensors which can be encompassed by this classification include those which can perform functions such as, but not limited to, those with the capability of detecting ambient audio patterns, detection of environmental changes, alterations and readings such as the temperature, humidity readings, altitude and overall pressure of the subjects location, etc. The data from this sensor may be collected from environmental sensors 139 upon the lie and emotion detection system, as well as solely or in conjunction with sensor systems 230, 330 connected via modular unit 210 or remote unit 310.

In some embodiments, as expressed in the above sections, the data which can be classified as sensor data can be obtained from locations connected, either by wired or wireless connections, with the lie and emotion detection unit 110 and classified as sensor systems 230, 330 located either upon the modular unit 210 and/or the remote unit 310. The nature of the system can be such that multiple sensors with similar and in some cases identical functionalities can be connected, for example a facial sensor 131 located on the lie and emotion detection unit 110 can be used in tandem with a facial sensor located on a remote unit 310. Alternatively, a sensor input can be connected to replace that upon the lie and emotion detection unit 110, for example if an ulterior sensor with additional and/or higher functionality can be utilized. In some embodiments, the infrared sensor 136 and/or the thermal sensor 137 can be connected to the system 100 through the connection of modular units 210 and/or remote units 310. The ability to utilize new and/or alternative sensors in the aforementioned manner allow the usage of the system 100 beyond the original maximum functionality of the original components. In some embodiments, the systems which encompass the sensor system 130 can omit any of those described above.

### Signal Conversion System

With reference to the aforementioned FIG. 1 discussed above, the system such as a lie and emotion detection unit 110 can include a signal conversion system 140, which when utilized can convert incoming signals into another routine. Analog and/or digital electrical signals can be converted into those more easily recognized by the user and/or subject using the signal conversion system 140, in some embodiments displaying the data information to the user and/or subject in real-time. These signals can be visual 142, audio 144, haptic 146, etc. The conversion of this system can also convert signals from an audio, visual, and/or haptic nature into those more readily processed by systems such as the processing system 120, and/or any other system used to process incoming data. As illustrated in the embodiment of one such system, the signal conversion system 140 can contain but is not limited to a visual component 142, an audio component 144, and a haptic component 146.

In some embodiments, the visual component 142 of the signal conversion system 140 can be realized in the form of a visual display device which can be designed to convert analog and/or digital signals into visual signals perceptible by the user and/or the subject. This can be realized in the following forms, with the understanding that the following list is not meant to be limiting, an OLED screen, an LCD screen, a projector, and/or any other display device. These display devices can be realized in a number of ways, such as upon the lie and emotion detection unit 110 or through a wired and/or wireless connection to a modular unit 210 and/or a remote unit 310.

In some embodiments, the visual images captured by the visual component 142 can be converted into analog and/or digital signals. For example, the image capture device can be a camera which can capture pictures and/or video from the user and/or subject. The visual components 142 can be connected to the system in such a way that it can be removed. This can allow the user and/or subject to remove the visual component 142 as desired. For example, the subject could attach a thermal camera to the system when in use, and remove it for day to day tasks. The ability to attach the visual components 142 of the system 100, as discussed herein, can be accomplished utilizing any of the but not limited to methods outlined and/or discussed in these embodiments.

In some embodiments, users of the system can be provided with visual data as desired through the use of the visual component 142. For example, the visual component 142 can give a visual representation of data collected from the system 100, such as the sensor system 130, to the user of the system 100. In this example, parameters of the sensors being utilized can be displayed to the user such as, but not limited to, the subject's heart rate, body temperature, vocal stress patterns, muscle movements, optical movements, thermal shifts, and/or such parameters and data. These visualizations can be displayed in a constant flow of data normally classified as real-time, and could also in some embodiments be used to display the status of the system, in addition to previous sessions of the subject or a selection of a population of subjects.

In some embodiments, visual data as presented to the user and/or subject of the system 100 can be such that the aforementioned visual displays relevant to the user and/or subject experience can be represented to the user and/or subject through the use of composite imaging, whereby the incoming video signal of the subject and/or user can be superimposed with relevant sensory and/or analysis data and presented to the user and/or subject in a adaptive composite manner. Example embodiments of the present system include, but are not limited to, vectoring information nearing the subjects ocular region, nasal cavity, and oral cavity, thermal imaging information, infrared imaging information, visual representations of the subjects voice and speech stress patterns, tracking of potentially irregular movements in the subjects facial and body movements, visual representations of the subjects physiological responses, visual cues indicating to the user and/or subject that the subject has told a lie, visual cues to the user the nature of the subjects emotional state, etc.

In some embodiments, a device such as a speaker can be utilized to convert analog and/or digital signals into sound waves for the benefit of the user and/or the subject of the system 100 through the use of the audio component 144. Some embodiments of this component can generate sounds waves from analog and/or digital signals. This could also be realized by capturing sound waves and converting into analog and/or digital signals, such as a microphone. In some embodiments, an audio component 144 such as an in-ear, on-ear, over-the-ear, and/or an outwardly facing speaker can be provided through the use of a modular unit 210 and/or a remote unit 310.

In some embodiments, users of the system can be provided with audio data as desired through the use of the audio component 144. For example, the audio component 144 can give an audible representation of data collected from the system 100, such as the sensor system 130, to the user and/or subject of the system 100. In this example, parameters of the sensors being utilized can be displayed to the user and/or subject such as, but not limited to, the subject's heart rate, body temperature, interference from outside sources, cancellation of said noise in some embodiments, detected stress on the subjects vocal patterns, and/or such parameters and data. These audio updates can be realized in a constant flow of data, and could also in some embodiments be used to reflect the status of the systems. An audio control component 144 can be used as a microphone which can be used in conjunction with operating the lie and emotion detection unit 110, modular unit 210, and/or remote unit 310.

In some embodiments, audio data as presented to the user and/or subject of the system 100 can be such that the aforementioned audio feedback relevant to the user experience can be represented to the user and/or subject through the use of multitrack layering, whereby the incoming audio signal of the subject and/or user can be superimposed with relevant sensory and/or analysis data, allowing for an adaptive composite homogeneous representation of audio information such as but not including the ability for the system 100 to give an audible cue to the user and/or subject when a lie is told, such as a tone, etc., audio associated with physiological data such as the ability to hear the subjects heart rate, etc.

In some embodiments, haptic data can be converted into analog and/or digital signals. For example, the haptic capture device can be an iWatch which can capture physiological data from the subject. The haptic component can allow the system 100 to provide sensory feedback to the subject when a lie is told. For example, the use of a modular unit 210 such as a mobile device could allow the system to provide feedback in the form of vibrations when a lie is detected. Users of the system can be provided with haptic data as desired through the use of the haptic component 146. For example, the haptic component 146 can give a tangible representation of data collected from the system 100, such as the sensor system 130, to the user of the system 100.

In some embodiments, component features and/or functionality as described above can be fulfilled by an modular unit 210 and/or a remote unit 310 to enhance the signal conversion system 140, or replace with signal conversion system 240, 340. For example, the visual component 142 of the lie and emotion detection unit 110 can be supplemented by a wired and/or wireless modular unit 210 and/or remote unit 310. This can allow the functionality and overall lifespan of the system to be increased, as it can adapt with new technologies as they are created.

Haptic data as perceived by the user and/or subject of the system 100 can be such that the aforementioned provides relevant tactile feedback to the user and/or subject through the use of combined signaling, whereby the incoming physiological signal of the subject can be combined with relevant sensory data, allowing for an adaptive composite homogeneous representation of tactile information such as but not including the ability for the system 100 to give a haptic response when the subject engages in deceit or states the truth, as well as different haptic responses when certain emotional responses are detected, such as unique tactile pulses for when anger is detected, etc.

### User Interface System

In continuous reference to the embodiment illustrated in FIG. 1, operation of the system 100 including the lie and emotion detection unit 110, modular units 210, and/or the remote units 310 can be regulated and/or administered by the user and/or subject through the usage of the user interface system 150. In some embodiments of the user interface system 150, the actions of the system can be conducted through the usage of one or more actuators 152 and/or one or more sensors 154.

In some embodiments, inclusion of switches mechanical in nature can be utilized in such ways including but not limited to, button, rocker, rotary and/or toggle switches. Parameters of the system 100 to be under the control of the user and/or subject include but are not limited to, a switch controlling the power to the lie and emotion detection unit 110, modular unit 210, and/or remote unit 310, brightness of any screens connected to the system 100, volume control of any audial systems attached to the system 100, etc. The usage of one or more actuators can be designed in such a way that the user and/or subject can alter the system 100 without directly viewing the actuators 152, performed in some embodiments through the usage of tactile feedback.

In some embodiments, resistive and/or capacitive sensors can be utilized as part of the sensor 154 aspect of the user interface system 150 allowing the system to detect contact from the user and/or subject, such as the users finger on a touch screen. The sensors 154 can be used in such a way that differing gestures on the touch screen can indicate an individualized action the user and/or subject wishes to perform, including but not limited to, taps in excess of two or three, tapping the screen in multiple locations, holding the screen in excess of specific second amounts, swiping in alternative patterns such as upwards, downwards, sideways left, sideways right, etc. An example of the usage of sensors as described above is in the selection of different composite sensor data information, which can in some embodiments be selected using, for example, a horizontal swipe to the left or right.

In some embodiments, component features and/or functionality as described above can be fulfilled by a modular unit 210 and/or a remote unit 310 to enhance the user interface system 150, or replace with user interface system 250, 350. For example, the actuator component 152 of the lie and emotion detection unit 110 can be supplemented by a wired and/or wireless modular unit 210 and/or remote unit 310. This can allow the functionality and overall lifespan of the system to be increased, as it can adapt with new technologies as they are created. This can also allow the user and/or subject to customize the feel, functionality, and/or usage of the interface to one that they prefer.

### Power System

Expressed in FIG. 1 is an illustrated embodiment of the system 100 such as a lie and emotion detection unit 110, which can be embodied by a unit including a power system 160, which can be designed to distribute energy to the system 100 including one or more systems of the lie and emotion detection unit 110, one or more systems of the modular unit 210, and one or more systems of the remote unit 310. In some embodiments of the power system 160, the actions of the system can be conducted through the usage of an energy storage component 162 and/or an energy generation component 164.

In some embodiments, energy which is to be utilized by the system 100, including the lie and emotion detection unit 110, modular unit 210, and remote units 310 can be stored/attained through the energy storage component 162. The embodiment can include a design in which there is a primary and secondary cell of a battery device, with the potential for one to be rechargeable or non-rechargeable. The storage capacity of the energy storage component 162 can vary based on the final embodiment of the system 100, for example the capacity could be a range of roughly 50 mAh to 500 mAH, a set amount of mAH, and/or other amounts as most applicable with the final embodiment of the system 100. Examples of potential energy storage component's include but are not limited to NiCad battery, Li-ion battery, Ni-MH battery, and a LiPo battery. Other embodiments of the energy storage component 162 include such devices as a fuel cell, capacitator, or other devices capable of storing energy.

In some embodiments, electric energy to be provided for the system 100 including the lie and emotion detection unit 110, modular unit 210, and/or remote unit 310 can be generated from differing sources, such as solar energy, electromagnetic energy, thermal energy, and/or kinetic energy, the conversion of which can occur through the usage of the energy generation component 164. In realizations of the system 100 whereby the embodiment makes use of the energy generation component 164, the system 100 perform such functions such as charge and run the system 100 wirelessly.

In some embodiments, component features and/or functionality as described above can be fulfilled by an modular unit 210 and/or a remote unit 310 to enhance the power system 160, or replace with power system 260, 360. The potential attachment of, for example, a remote unit which includes a energy generation component 164 can increase the functionality of the system and allow the user and/or subject an increased duration in which the system 100 can be utilized. Some embodiments of the system 100 are such that the power system 160 can be omitted by the system 100.

### Input/Output (I/O) System

With reference again to the system 100 illustrated in FIG. 1, one or more modular units 210 and/or remote units 310 can interface with the lie and emotion detection system unit 110 of the system 100 via an I/O System 170. In some embodiments, this system can be designed to have wireless connections to these other systems, and/or the potential for wired connections, such as ports and/or connectors, to allow coupling by the system 100. As demonstrated in FIG. 1, one embodiment of this system 100 can allow the lie and emotion detection unit 110, one or more modular units 210, and/or one or more remote units 310 to communicate with each other by utilizing the applicable I/O system 170. These communications can be initialized by any of these systems and received by any other system. For example, the remote unit 310, such as a camera, can communicate with the lie and emotion detection unit 110, while the lie and emotion detection unit 110 communicates with the modular unit 210, and any or all other potential communicators herein.

In some embodiments, the wireless system can be comprised of one or more receivers 174 whereby signals can be obtained by the system 100, and transmitters 176 whereby wireless signals can be delivered by the wireless system to other systems. In some embodiments, transceivers can be included, the performance of which allow tasks similar to both those of the receivers and transmitters to be undertaken. The process of receiving and transmitting can in some embodiments be performed through the utilization of antennas, which can receive electric signals including but not limited to ANT, ANT+, Bluetooth, Bluetooth Low Energy also known as Bluetooth Smart, MMS, Wi-Fi, CDMA, Zigbee, and GSM, and/or any other type of signal.

In some embodiments, protocols can be utilized to execute the wireless communication between one or more receivers and/or one or more transmitters. The process can include but are not limited to ANT, ANT+, Bluetooth, Bluetooth Low Energy also known as Bluetooth Smart, MMS, Wi-Fi, CDMA, Zigbee, and GSM. The process can be executed such that the lie and emotion detection unit 110 can be designated ANT+ master unit with regard to other ANT devices. The one or more receivers, one or more transmitters, and/or one or more transceivers do not have to be limited to one of the above protocols, which allows the system to implement a larger scope and/or breadth of additional systems to be received by the lie and emotion detection unit 110. In some embodiments, signals can be obtained by the receiver via a global positioning satellite, also referred to as GPS. The ability of the wireless system to communicate with the any and all modular units 210 and remote units 310 associated with the system 100 is demonstrated in the illustrated embodiment.

In some embodiments, mechanical and/or electronic coupling to the lie and emotion detection unit 110 of systems such as modular units 210 and/or remote units 310 via ports and/or connectors can be implemented to facilitate the process of wired communication. The process can include such connectors as the following: a Universal Serial Bus (USB) port and/or connector, such as USB 1.0, USB 2.0, USB 3.0, USB 3.1, with the possibility of including such devices as an IEEE 1394 (FireWire) port and/or connector, a Display port and/or connector, microUSB and type-C ports and/or connectors, an HDMI port and/or connector, an Ethernet port and/or connector, a coaxial port and/or connector, a Thunderbolt port and/or connector, an optical port and/or connector, a DVI port and/or connector, and or any other ports and/or connectors which would be suited to the operation of the system 100. The system can be designed in such a way that a multitude of different ports and/or connectors are present upon the lie and emotion detector unit 110, broadening the scope of available wired connections which can be made to the system 100. For example, one such port could be a USB port while another could be a HDMI port. The potential for mechanical and/or electronic coupling of the lie and emotion detection unit 110 to the remote units 310 associated with the system 100 is demonstrated in the illustrated embodiment.

In some embodiments, the outward appearances of the modular units 210 and/or remote units 310 can vary vastly amongst differing modular units 210 and remote units 310, along with supported features each modular unit 210 and/or remote unit 310 contributes to the system 100. The internal configurement of mechanical and/or electronic systems can remain similar, allowing the user and/or subject to alter the modular units 210 and/or remote units 310 connected to the preference of the user and/or subject. In some embodiments, a variable selection of modular units and/or remote units can be made available to the user and/or the subject to customize the lie and emotion detection unit 110 to personal preference, allow for outdated units to be disconnected and replaced as applicable, and/or allow damaged units to be replaced without necessitating the purchase of a new system 100 in its entirety. The modular units 210 and remote units 310 as outlined above can in some embodiments include connections such as a USB connector, or a connector which similarly allows for connections to be made for a large variety of electronic devices. For example, the presence of such a connector allows the user and/or subject to connect the modular units 210 and/or remote units 310 to devices such as but not limited to a mobile device or computing device. In some embodiments, such modular units 210 and remote units 310, can be coupled such that the two units can form a compact unit more readily managed by the user and/or subject.

### Deception and Emotion Interpretation and Analysis

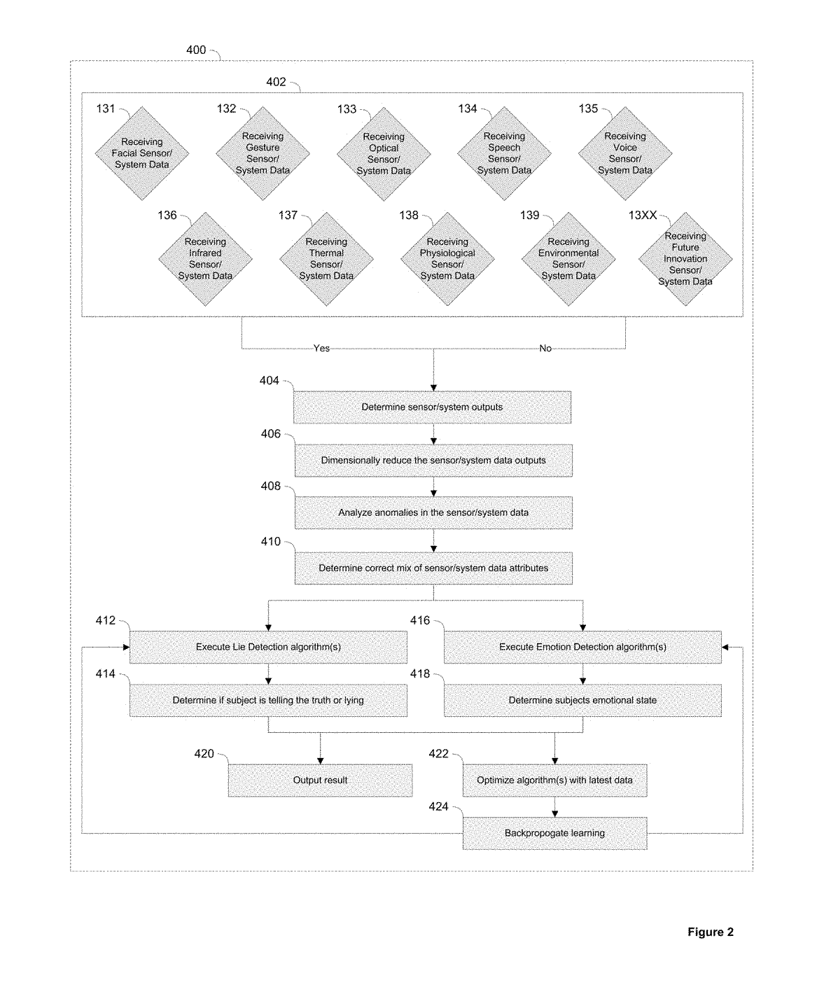

With reference to the embodiment of logic within the lie and emotion detection system 110 illustrated in FIG. 1, the process of receiving and analyzing sensor data 402 is illustrated in FIG. 2. The following is a potential actualization of a system where incoming sensor data 402 can be implemented, whereby the incoming sensor data will be weighted and by which the system can determine if the threshold for validity has been reached. Following this step, the system 400 can analyze against overlying norms as well as individual reactions of the subject using algorithmic analysis to determine if the subject is engaging in deceit, or the emotional state of the subject, as applicable. This deception and emotion analysis process can be realized in the form of several interconnected steps, described below. In alternative embodiments, fewer, additional, and/or different steps may be performed. Also, the use of a flow diagram is not meant to be limiting with respect to the number or order of the steps performed.

In some embodiments, the deception and emotion detection analysis 400 can first begin with sensor data 402 being collected by the system. This can occur through the use of existing devices present, or can be used by the connection of a modular unit 210 and/or remote unit 310, such as a camera or microphone. Potential inputs include but are not limited to facial sensor/system data 131, gesture sensor/system data 132, optical sensor/system data 133, speech sensor/system data 134, voice sensor/system data 135, infrared sensor/system data 136, thermal sensor/system data 137, physiological sensor/system data 138, environmental sensor/system data 139, as well as any and all sensor/system data relevant to the system 100, 400 which have not yet been discovered and/or created 13XX. The ability for a sensor input to be used towards the model may also be determined by the presence of environmental interference present at the subjects location. The sensor data may also be used to collect data which will further inform on the subjects behavior in order to reduce false positives.

In some embodiments, the presence of these inputs 402 can then be used to determine the applicability of said inputs 404 being utilized for each session. The process by which inputs are determined can be performed by the system itself with limited user interference, which can aid in the removal of human bias in the deception and emotion analysis system 400. This process can also include visual cues to the user, by which the subject can troubleshoot potential issues with equipment. Upon the determination of incoming sensor inputs 404, the system 400 can also be receiving too few inputs to reliably give an accurate deception or emotion result, based on threshold input values which are determined based upon the weighted average of inputs being received. For example, if the only input to be received by the system is voice stress sensor/system data 135 and the threshold value needed is at least three inputs being received, a visual and/or audio cue can be shown to the user and/or subject that additional inputs must be enabled and/or connected, or the system cannot give an accurate result.

In some embodiments, the previously determined sensor outputs 404 will then undergo the process of multifactor dimensionality reduction 406, which can be used to maximize the contribution of each individual sensors data 402 to the aggregated model. The arrangement of the data within the processing system 120 can link relevant data points together, such as the increase in microtremors in the subjects voice as they undergo an increase in stress with pupil dilation to substantiate the finding. Depending on the utilization of the system, this could be used to determine if the subject is attempting to engage in deception and/or the potential for a mood shift to one more commonly thought to be negative. In the latter case, the user could then redirect the conversation away from a potentially sensitive topic to the subject. Another step that can be implemented in the process of multifactor dimensionality reduction 406 is the process of segmentation via machine learning, which can include the ability for the system to further process the raw sensor data 402 into data which can be referenced against other differing methods of data. For example, the visual data 142 obtained from the facial sensor/system data 131 and the audio data 144 obtained from the speech stress sensor/system data 134 can be transformed into a unit of measure so that the two inputs can be utilized comparatively. Additionally, utilization of canonical-correlation analysis can benefit the maximization of contribution levels in relation to sensor data. For example, canonical-correlation analysis can be employed to produce a model equation for a plurality of explanatory measures and a plurality of performance variables. This process can also be augmented through the use semantic mapping.

In some embodiments, the deception and emotion analysis system 400 can next take the multifactor dimensionality reduction 406 and assess the contribution the sensor output will have upon the model. By analyzing anomalies in the sensor/system data 408 the system can determine the current weight vector for a given sensor against that sensors ideal weight vector. In some instances, this evaluation can occur based on the environmental sensor data 139 collected and the effect this can have on the sensor data. For example, if the subject is currently located somewhere with a substantial amount of background audio interference, the potential for the voice stress sensor data 135 and speech stress sensor data 134 may be compromised. Another potential contribution to interference can arise from the subject themselves. For example, a subject wearing eyeglasses has the potential to interfere with the ability of optical sensor data 133 to be collected from the subject.

In some embodiments, there can be a method by which the determination will occur for the level of contribution each sensor will have upon the model as a whole 410. For instance, if there are very few adverse environmental impacts upon the sensor data and thus sensor data is being received very clearly, it will fall close to the ideal weight vector for the respective output. In this instance, the sensor output will have a high contribution level to the algorithmic models. Alternatively, if there are substantial limits to the sensor data output due to interferences assessed 408 the current weight vector can differ from the ideal weight vector, and thus the contribution to the model will be limited. In some embodiments, this method of determination can be realized through the use of decision tree analysis. In some embodiments, the process of analyzation can include the use of nonlinear dynamic models and artificial neural networks, which will be able to learn in limited areas as related to the system 400. In some embodiments, multi-layered adaptation allows the utilization of new data upon existing interview data, for a specific subject(s).

In some embodiments, the method of execution of the algorithm(s), to determine whether the subject is currently engaging in deceit 412, or alternatively the determination of which emotion is the most likely to be expressed by the subject 416. The most suitable algorithm can vary based on the sensor data being received 402 and other potential tools for determination, such as stored previous sessions of the same subject, or subjects that fall into the same population as the subject. A few algorithms which can be utilized are detailed in the following, with the understanding that this is not meant to be limiting and that other algorithms may be utilized by the system.

In instances of sensor data that has complexity ranges which will allow it, the use of Fast Fourier Transform algorithms can be utilized. An example of sensor data which could be suitable is voice stress sensor data 135 and speech stress sensor data 134, whereby determination of the decoded audio power in the stressful frequency range is more powerful than that of the normal range. The potential complexity of visual image data patterns can necessitate the use of an adaptive artificial neural network to accurately describe the non-linear data set and to aid in the processing of the discrete mathematics involved in a vector based program as contained in the Processing System 120. A potential sensor data which can utilize this algorithm is the facial sensor/system data 131. A K-means classification clustering algorithm can be utilized to help find correlations between subjects which can lead to the discovery of certain similarities across these individual subjects and rank them according to the degree of similarity. This can help inform and create another tool for the user. A hypothetical example which could be discovered is that the presence of a large amount of makeup upon the subjects face could be linked to the probability of the subject to display increased microtremors when compared to other subjects. In cases whereby the subject has previously undergone this process, the system can access memory 124 and storage 128 to utilize the historical data collected and take advantage of a naïve bayes algorithm for probabilistic classification. The accuracy of the current session can thus be increased by determining prevalent actions of the subject. An example for the usage of this algorithm is in identifying individual expressions, or ‘tells’, of the subject, such as itching upon the nasal region when the subject engages in deceit. While this in and of itself is not a prevailing determination for deceit, for a specific subject this could be discovered using such algorithms and in the analysis of historical session data. In other instances, a logistic regression algorithm can be utilized to predict the outcome of a categorically dependent variable. One potential sensor data point which can utilize this method is thermal sensor data 137. The use of regression analysis can be beneficial by the application to disparate algorithms, against each input and to the overall model.

In some embodiments, the execution of the program 126 by the user will allow the utilization of the aforementioned algorithms 412, 416 with the desirable outcome analyzation of the incoming data to allow the user further insight into the subject. This insight can allow the user detection of deceit 414 when presented with stimuli such as a presented question. Alternatively, the same methods can predict that the subject has relayed the truth in response to stimuli 414. The emotional state of the subjects can additionally be determined in response to stimuli 418, for example their instinctual response to news being relayed by the user to the subject. It can be conceived that these two processes can also be performed in parallel, allowing for both the deception and emotion analysis to be utilized simultaneously. Upon the determination, the user and/or subject will be displayed the result 420 in either an audio 144, visual 142, or haptic 146 manner, and can thus proceed with the session as best determined by the user. In some embodiments and when certain thresholds are met, the determination can be displayed to the subject in a visual 142, audio 144, and/or haptic 146 manner, for example if a certain threshold is met determining the presence of a lie the subjects device, such as a cell phone, can provide haptic 146 feedback, such as the vibration of the device, and may deter the subjects from the continual usage of deceit in the interaction with the user. The system will also use this determination to optimize the performance of predictive modeling 422 based on the most current data. For example, the determinations of this line of questioning can then be used in a random forest algorithm by building a forest of many decision trees over different variations of the same data set and then taking the weighted averages of the results. This technique could aid in predicting a questioning technique because it can effectively identify patterns across a large and oftentimes noisy dataset.

In some embodiments, upon the successful optimization update of algorithms 422 based on the most current utilization of the system, the process of backpropagation 424 can occur as a method of keeping the neural, or in some embodiments bayesian network(s), or in some embodiments algorithm(s), most effective. This process will utilize the entire scope of weight vectors relevant to the session, and is used to optimize their current weights in an effort to minimize the loss function of the system and to reduce/eliminate false positives.

Certain embodiments of the present invention include, but are not limited to combining multiple control techniques, (e.g., neural network control, non-linear control . . . ) into overarching adaptive algorithms for multifactor dimensionality reduction, anomaly detection, and prediction for the systems comprising 100. The system contains an attribute extractor program for extracting an attribute weight vector, wherein the current attribute weight vector contains information related to audio, visual, and physiological data. A machine learning model generation program may be utilized for generation of a classification model from the current attribute weight vector, a plurality of data functions and a certainty function vector wherein the classification model associates the information of the current attribute weight vector and the ideal state certainty function vector with patterns and each of the plurality of patterns is associated with a respective one of a plurality of attribute classifications. A certainty function generating program for generating a certainty function based on the classification model and the current attribute weight vector can be used by the system, wherein the certainty function contains information representing a likelihood of each attribute belonging to a respective one of the plurality of classifications. A contextual attribute extractor program can be utilized for the extraction of the certainty function attribute vector from a previously generated certainty function, wherein the certainty function attribute vector contains information related to audio, visual, and physiological data of the certainty function, wherein the classification model is updated to iteratively improve the classification models based on the latest extracted certainty function attribute vector and further wherein both the certainty function attribute vector is extracted and the classification model is updated, for a threshold number of iterations. Additionally, a machine learning classification program for the classification of attributes of a secondary audio, visual, and physiological attributes based on the classification model can be utilized.

### Lie and Emotion Detection Interfaces

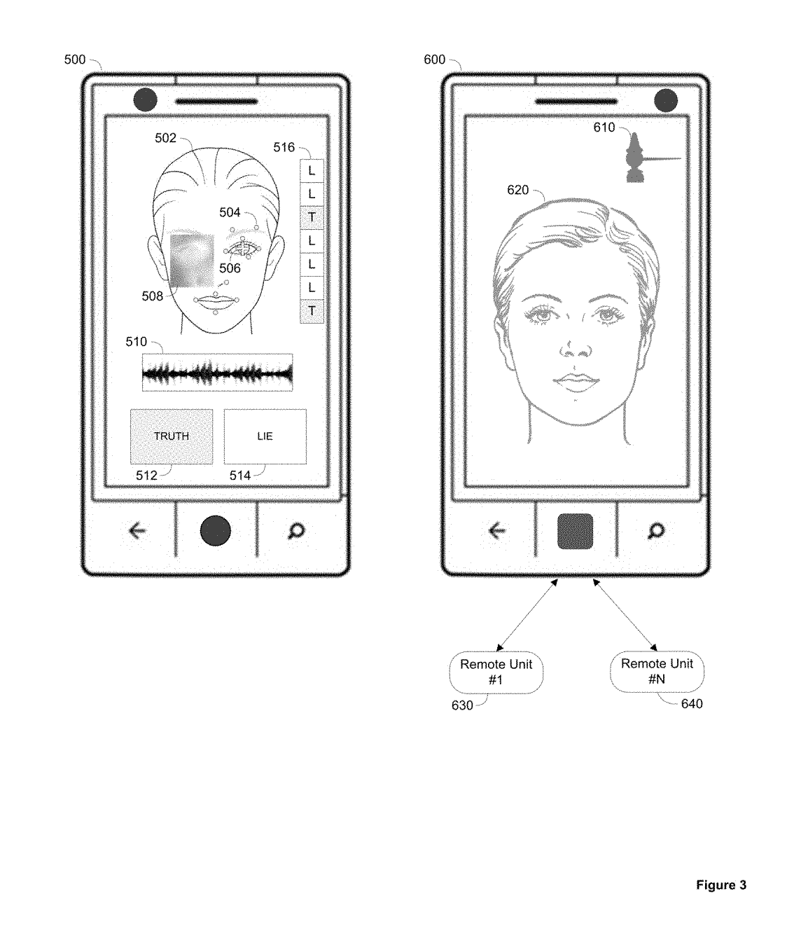

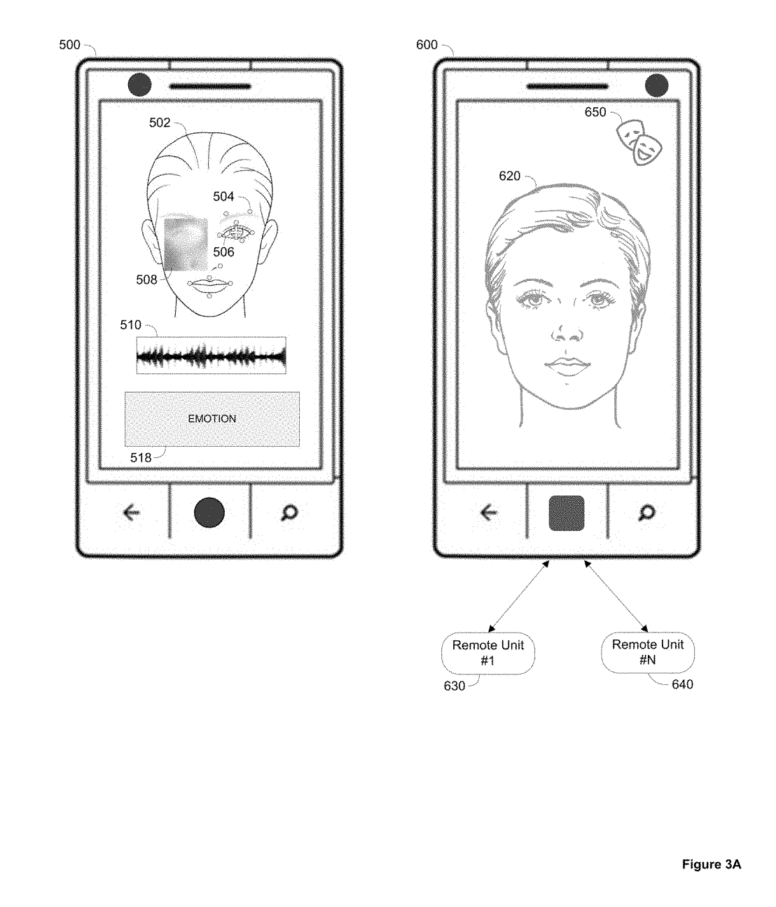

With reference now to the embodiment of the electronic device or system 500, such as a lie and emotion detection unit or emotional analysis system. The ability of the system 500 to receive, process, and relay data to the user of the system 620 is illustrated in FIGS. 3 and 3A. In some embodiments, the system 500 can include several remote units 630, 640 which can be removably coupled to the lie and emotion detection system of the subject 600. The lie and emotion detection unit 500 and the remote unit(s) 630, 640 can be similar to, or the same as, the lie and emotion detection unit 110 and/or modular unit 210 and/or the remote unit 310 as previously discussed in detail with regards to FIG. 1. The visual interface of the user 620 can include but is not limited to such data as described as follows; a representation of facial sensor/system data 131, gesture sensor/system data 132, optical sensor/system data 133, thermal sensor/system data 137, infrared sensor/system data 136, speech stress sensor/system data 134, voice stress sensor/system data 135, physiological sensor/system data 138, environmental sensor/system data 139, and future innovation sensor/system data 13XX. The interface can also provide auditory, tactile and visual confirmation that the subject 502 is engaged in deception or speaking the truth, or a visual display of the subjects 502 current emotional state. The system of the subject 600 can include but is not limited to a visual representation of the user 620, and a visual confirmation for the subject that the system is being utilized.

In some embodiments, the visual representation of the subject 502 can provide the user with information regarding the current use of deception by the subject in real time. In regards first to the lie and emotion detection system, the system can give visual confirmation of the subject's engagement of deception, lighting up truth 512 or lie 514 as applicable. It is conceivable that the method of confirmation can be altered by the user, for example if the user would prefer an audible tone or tactile feedback when the subject 502 engages in deception. The system can also provide a history of the session 516, which can allow the user insight into the subject's overall use of deception based on the questioning technique. For the emotional analysis component of the system, these features can be replaced and instead show the current emotional state of the subject 518. In some embodiments, some of these features can be hidden by the user, allowing for customization of the interface by the user. In some embodiments, the two operating modes can be used in tandem, allowing the user 620 to determine, for example, the emotional reaction of the subject if the user discloses they know a lie was told.

In some embodiments, the system can include automated feature and orientation tracking for geometric extraction and recognition 504, spanning the subjects entire cranium including, but not limited to; the crescentic line of hairs at the superior edge of the orbit, the ocular region, the nasal region, the oral cavity region including labium, pinna, and mentum. This can allow the analysis of involuntary muscle movements in the subject, including personal movements associated with certain situations. In these instances, the presence of previous recordings can better inform on the subjects individual responses along with prevailing movements. This data can be captured using present video or in some embodiments, remote units 630, 640 can be utilized. For a more detailed description of what is captured using the facial sensor/system 131, see sensor system outlined previously. This interface can be customizable by the user, with this visual feature being hidden if desired and performing solely in the background of the system.

In some embodiments, the system can include automated optical scanners 133 for feature and orientation tracking for geometric extraction and recognition 506 spanning the entire bulbus oculi, including, but not limited to; the pupil, iris, and sclera. This can allow the analysis of minute changes in the ocular orientation of the subject, such as dilation of the pupil in response to posed questions or information. This data can be captured using present video or in some embodiments, remote units 630, 640 can be utilized. For a more detailed description of what is captured using the optical sensor system 133, see sensor system outlined previously. This interface can be customizable by the user, with this visual feature being hidden if desired and performing solely in the background of the system.

In some embodiments, the system can include data relayed from a thermal sensor 137 or infrared sensor 136 as depicted 508. This can include the thermal map of the subject, as well as information such as blood flow analysis. This data can be obtained from remote units 630, 640, which in some embodiments can include an attachable thermal and/or infrared camera. For a more detailed description of what is captured using the thermal sensor system 137 and infrared sensor system 136, see sensor system previously outlined above. This interface can be customizable by the user, with this visual feature being hidden if desired and performing solely in the background of the system.

In some embodiments, the system can include physiological data from the subject 502. This can include readings such as heart rate. This data can be obtained from remote units 630, 640, which in some embodiments can include a physiological monitor such as a FitBit. For a more detailed description of what is captured using the physiological sensor system 138, see sensor system outlined above. This interface can be customizable by the user, with this visual feature being hidden if desired and performing solely in the background of the system.

In some embodiments, the system can include audio analysis of the speech 134 and voice stress 135 of the subject 510. The user can be relayed changes in the pitch of the subject, or microtremors present in the voice which can be present during stress. This can also relay changes in the diction of the subject. This audio data can be recorded from any microphone, including a remote unit 630, 640 which can be attached to the subjects computing device 600. For a more detailed description of what is captured using the voice stress sensor system 135 and speech stress sensor system 134, see sensor system outlined previously. This interface can be customizable by the user, with this visual feature being hidden if desired and performing solely in the background of the system.

In some embodiments, the subjects system 600 can include a visual representation of the user 620. In instances where the emotional analysis component is being utilized, in some embodiments the subject will be notified by the presence of an associated symbol 650 which will inform the subject 620. In reference to the lie detection system, the subject can also be made aware of the use of the lie detection software by the presence of an associated symbol 610. The system can also inform both the subject 620 and user 502 when a blatant lie is told, the parameters of which can in some embodiments be specified by the user at the start of the process, or set to a default. In some embodiments, the user can be notified by visual representation such as a pulse across the visual interface, and/or a darkened history display 516. In some embodiments, the presence of a blatant lie will also be relayed to the subject 502. This can include visual representation such as the alteration of the lie detection system symbol 610, i.e. the growth of Pinocchio's nose. The notification could also be an audible note or tactile feedback. The purpose of this feedback would be to discourage future lapses in truth from the subject.

In some embodiments, potential users such as law enforcement operations could focus primarily on the lie detection aspect of the system. However, the potential application of the somatic marker hypothesis could increase the validity of the emotional state aspect of the system. The hypothesis states that somatic markers link certain physiological responses to decisions which can create a bias of an individual to make a certain choice regarding that decision. A potential application this allows is in the field of medicine, whereby a doctor could determine if a patient has a substance addiction by using the system to record the emotional reaction, in particular the physiological response of the subject, when exposed to stimuli.

### Network Communication

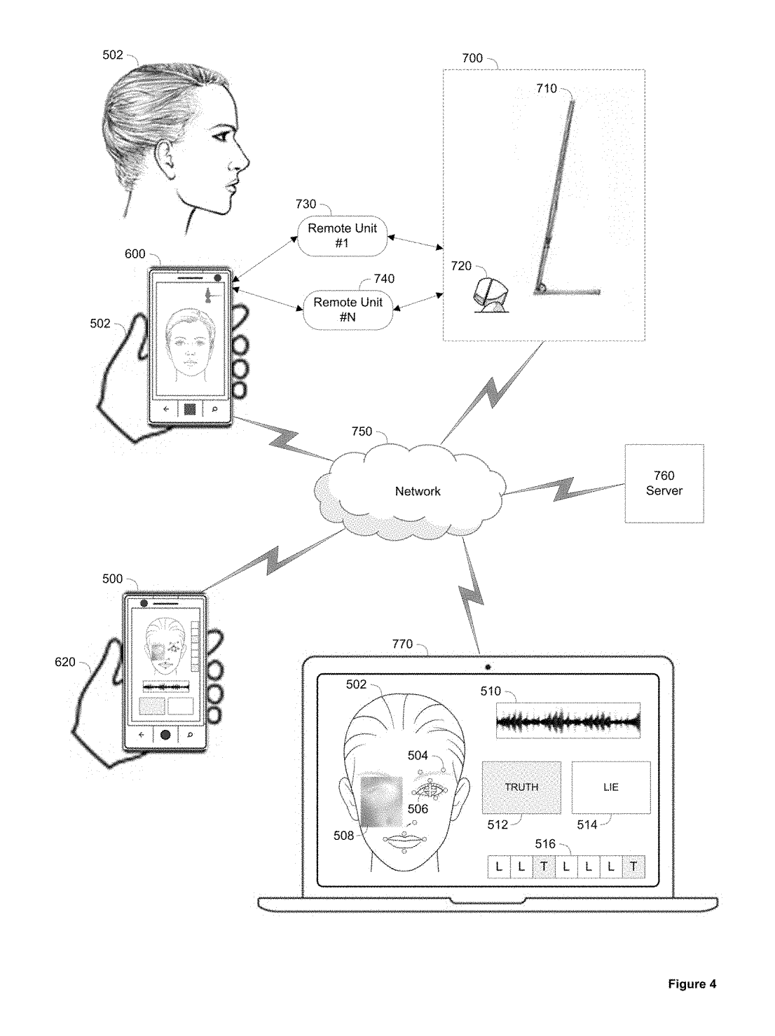

With reference to communication between the various aspects of the application of lie and emotion detection as demonstrated by FIG. 4, in some embodiments, the network 750 can access information from the lie and emotion detection system on whichever computing device it is being utilized from, and then transfer from the device 700 to the server 760. Alternatively, the server 760 can communicate via the network 750 with the computing devices of a plurality of users 620, 770 to relay information on, in some embodiments, the plurality of subjects 502, as well as the potential to access needed functions. The process by which the client server 600, 700 connects to the network 750 and thus the server 760 may be one of request and response.

In some embodiments, the subject 502 can be accessed by the lie and emotion detection system 700 through the use of a computing device, such as a computer 710 or mobile device 500. The video and audio capture of this computing device can be supplemented through the presence of remote units 720, 730, 740 which can in some embodiments include but are not limited to a thermal imaging camera, infrared imaging camera, microphone, camera, a device used to capture differences in temperature of the environment such as a thermometer, and/or a physiological monitor such as a FitBit, connected either wired or wirelessly to the lie and emotion detection system 110. The lie and emotion detection system 700 can then be accessed by the network 750 via wired or wireless connection. Once connected to the network 750 the system can transfer and/or receive data securely and unsecurely from the server 760. This can aid the system in detecting mannerisms unique to the subject 502. If there is no previous data for a subject 502, the server 760 and/or lie and emotion detection system 110 can create a log for the subject 502 to be utilized in the present and future sessions.

In some embodiments, the process of request and response can begin with the client server, e.g. the subject's computing system 600, 700, which can transmit a request via the network 750 to the server 760. In some embodiments, a response will be relayed from the server 760 to the user(s) 620 via their computing device 500, 770. An example of what could be transferred in this manner is legacy data, or previous session information of the subject 502 or quantitative population information related to the subject(s). Another potential use of the ability to pull information is the ability to utilize a thin client, or a method by which the software utilized is kept on the server 760, and execution of algorithms 412, 416 discussed in FIG. 2 is performed within the server 760 and the client system 700 is used as an access point, with the results being transferred to the user(s) 620. In some embodiments the software will be installed directly onto the client system 700 and all information on the implementation of the program located on the computing device 710 and the results transferred to the user(s) 620. In some embodiments of the system, the backpropagation learning process 424 by which algorithms will be enhanced will be transmitted to the server 760 via the network 750. In some embodiments, the data of each session can be stored locally on the client system and also located on the server 760.

In some embodiments, in addition to access by the server 760, the user(s) 620 lie and emotion detection system will also access that of the subject(s) 700, 600. This data can be accessed by the user through the use of a computing device, such as a laptop 770 or mobile device 500. The visual data from the subject will be used to display composite inputs such as: a visual representation of the subject, automated feature and orientation tracking for geometric extraction and recognition spanning the subjects entire body and cranium 504, automated optical scanners for feature and orientation tracking for geometric extraction and recognition spanning the entire bulbus oculi 506, automated thermal and infrared imaging 508, and audio capture for the purpose of voice stress and speech stress analysis 510. It is conceivable that the composite data information being displayed to the user can be viewed in such a way that multiple data streams can be viewed separately, for example the user can swipe horizontally to change the view from facial recognition tracking to thermal imaging information, etc. The history toolbar 516 may be stored and populated via the processing system 120 of the computing device for the duration of that session.

In some embodiments, the process by which complex data sets are transferred, stored, and processed is that of the concept of big data from the client system to the server 760 via in one embodiment an event queue. This process can begin with event generators, such as scripted actions, business rules, and workflow designated by the system. The event queue can then determine the most efficient way to relay the data to the servers, creating a record of these generated events when relayed. This method can allow the ability to access the majority of the system, such as the algorithmic information, allowing the servers to be remotely accessed by the client system. Scripted actions can be specified to notify the user 502 in the event that the subject engages in deception, or the determination of the subjects emotional state. This process can also allow the user access to the overall results of the session and population information.

In some embodiments, the information derived from the above processes can be utilized comparatively within a group of subjects, for the life time of said subject(s). Extracting a plurality of contextual attribute vectors further comprises the characterization by the system a plurality of the audio, visual, and physiological data of a respective one of the plurality of the certainty functions with features based on the likelihood of previously captured audio, visual, and physiological data belonging to a respective one of the plurality of classifications associated with the respective one of the plurality of certainty functions. The user can select a certain population which the user has an interest in the comparison of, for example an employer attempting to gain knowledge of the overall happiness and/or honesty of his/her employees. This method can give a historic graphical output of the average of the population selected; additionally, it can provide the current average trend of the population which can allow the user to gain an understanding of the alterations in the populations. In the aforementioned example, the employer might see that after a meeting, the employees had begun to be more truthful. Alternatively, if after an announcement the happiness of the employees had decreased the employer might attempt a morale boosting course of action.

In some embodiments, the server data can also be accessed by the user to revisit previous sessions. In some embodiments, early recordings can be reprocessed using the most current data on the subject. In some embodiments, this can eliminate the necessity of repeated lines of questioning.

In some embodiments, connections between any and all systems as described above as related to the system 100 including connection between the users devices 500, 770, the subjects devices 600, 710, remote units attached to the system 720, 730, 740, and the server 760 can be achieved using secure connections utilizing methods such as data encryption. Requests for the transference of information can utilize in some embodiments a Hypertext Transfer Protocol Secure (also referred to as HTPPS) or a Hypertext Transfer Protocol (also referred to as HTTP) request, wherein the response will be of the corresponding request type. For example, a request made utilizing a Hypertext Transfer Protocol Secure (HTPPS) request would garner a corresponding HTPPS response. Security measures can also be utilized to encrypt and thus protect data stored on servers, devices, etc. including previous subject information as well as in some embodiments the protocols and methods governing the system. In some embodiments, this can include the use of encryption keys and/or the ability to encrypt specific instance fields or attachments using AES128 or AES 256.

What is claimed is:

1. An electronic lie detection and emotional analysis system, comprising:
a computing system whereby the system facilitates the transfer of audio, visual, and physiological data from a subject through the utilization of an input/output system comprised of wired and/or wireless connections and at least one of the following components:
a processor;
a memory;
a sensor;
a signal converter;
a receiver configured to wirelessly communicate with at least one remote unit;
a transmitter configured to wirelessly communicate with at least one remote unit;
a transceiver configured to wirelessly communicate with at least one remote unit;
wherein the input/output systems of the lie detection unit and the extensible unit are configured to provide a wired electrical connection between the lie detection unit and the extensible unit when in a coupled configuration via the at least one wired connection the lie detection unit and the at least one wired connection of the extensible unit

2. An extensible system, per claim 1, wherein an extensible unit by which the usage of will allow for a or to supplement existing systems;
the here aforementioned system comprises at least one of the following components:
a processor;
a memory;
a sensor;
a signal converter;
a receiver configured to wirelessly communicate with at least one remote unit;
a transmitter configured to wirelessly communicate with at least one remote unit;
a transceiver configured to wirelessly communicate with at least one remote unit;

3. The electronic system according to any of claims 1-2, wherein the input/output system of the extensible unit comprises a first wired connection and a second wired connection, where at least one of the first and second wired connections are configured to couple a second extensible unit to the first extensible unit and to provide communication between the lie detection unit and the second unit extensible unit.

4. The electronic lie detection and emotional analysis system according to claim 1, wherein: the extensible unit comprises the sensor, and the extensible unit comprises the receiver and the transmitter and is configured to serve as a node for wireless communication with multiple remote units.

5. The device as recited in claim 1, wherein said system is further operable to execute code for: providing secure communications; and providing selected encryption information items to said transmission systems.

6. The method of claim 5, further comprising: sending, from the system in claim 1 to the extensible unit or remote unit or server, a request for encrypted communications, whereby sending the request to transmit includes: sending the request to communicate via a Hypertext Transfer Protocol Secure (HTTPS) request or a Hypertext Transfer Protocol (HTTP) request, where sending the request for the encrypted communication includes: receiving the request for encrypted communication via a HTTPS request or a HTTP request, where transmitting the encrypted communication includes: transmitting the encrypted communication as a HTTPS response or a HTTP response.

7. A method of analyzing multiple sources of the subjects audio, visual, and physiological information regarding lie detection analysis and emotional analysis according to any of claims 1-4, comprising:
the system of claim 1, wherein the execution of code whereby the audio, visual, and physiological information will be evaluated to determine the validity, usefulness, and/or highest contribution to the appropriate system;
analysis of incoming audio, visual, and physiological data for indications of lying and the emotional state of the subject by comparing the audio, visual, and physiological attributes of the subject to previously stored expression and/or parameterized value;
wherein, the system of claim 1 predicts if the subject is being truthful or lying;
wherein, the system of claim 1 predicts the subjects emotional state;
the system of claim 1, generates a composite visual, audio, or haptic response to the user indicating if the subject is being truthful or lying;
the system of claim 1, generates a composite visual, audio, or haptic response to the user indicating the emotional state of the subject;
the system of claim 1, may provide a composite visual, audio, or haptic response to the subject indicating if the subject is being truthful or lying;

8. The systems and methods of claim 7, wherein the audio, visual, and physiological information are generated and presented in real-time.

9. The system of claim 1 contains an attribute extractor program for extracting an attribute weight vector, wherein the current attribute weight vector contains information related to audio, visual, and physiological data;
a machine learning model generation program for generating a classification model from the current attribute weight vector, a plurality of data functions and a certainty function vector;
wherein the classification model associates the information of the current attribute weight vector and the ideal state certainty function vector with patterns and each of the plurality of patterns is associated with a respective one of a plurality of attribute classifications;
a certainty function generating program for generating a certainty function based on the classification model and the current attribute weight vector, wherein the certainty function contains information representing a likelihood of each attribute belonging to a respective one of the plurality of classifications;
a contextual attribute extractor program for extracting the certainty function attribute vector from a previously generated certainty function, wherein the certainty function attribute vector contains information related to audio, visual, and physiological data of the certainty function, wherein the classification model is updated to iteratively improve the classification models based on the latest extracted certainty function attribute vector and further wherein both the certainty function attribute vector is extracted and the classification model is updated, for a threshold number of iterations;
a machine learning classification program for classifying attributes of a secondary audio, visual, and physiological attributes based on the classification model.

10. The method of claim 9, wherein extracting a plurality of contextual attribute vectors further comprises: characterizing by the system a plurality of the audio, visual, and physiological data of a respective one of the plurality of the certainty functions with features based on the likelihood of previously captured audio, visual, and physiological data belonging to a respective one of the plurality of classifications associated with the respective one of the plurality of certainty functions.
